(19) United States
(12) Patent Application Publication
(10) Pub. No.: US 2012/0209145 A1
(43) Pub. Date: Aug. 16, 2012

(54) MEDICAL SUIT OF AXIAL LOADING WITH AUTOMATIC CONTROL SYSTEM

(76) Inventors: Anatoliy Ivanovich Grigoriev, Moscow (RU); Inesa Benediktovna Kozlovskaya, Moscow (RU); Umberto Orazio Giuseppe Maugeri, Calvignano (IT); Oleg Igorevich Orlov, Moscow (RU); Irina Valerievna Sayenko, Naro-Fominsk (RU); Yevgeniy Petrovich Tikhomirov, Moscow (RU); Yevgenia Nikolaevna Yarmanova, Zhukovskiy (RU)

(21) Appl. No.: 13/261,272

(22) PCT Filed: Oct. 22, 2010
(86) PCT No.: PCT/EP2010/065968

§ 371 Date: Apr. 20, 2012

(30) Foreign Application Priority Data
Oct. 22, 2009 (RU) 2009138820

(51) Int. Cl. A61B 5/103

(52) U.S. Cl. 600/587

(57) ABSTRACT

The area of application of the invention is the development of special cloths for treatment in clinical conditions of the motor function disorders at child's cerebrally palsy, disturbances in vascular system of brain (strokes), and other diseases followed by disturbances of motor coordination both of arms and legs, and also for measuring and registration of loads on patient body created by the suit. The suit contains coverall made of one (at least) fabric layer, and tensioning device. The tensioning device provides the creation of axial loading on the musculoskeletal system with use of buffers. The coverall is additionally provided with the system of loads measurement and registration containing power source, tensodynamometers with intensifiers placed in the fixed anatomic points of cloths, and analog-digital converter with USB cable, so the system converts analog signals into digital ones and transfers them to computer for operative analysis, imaging and registration. Each buffer has the strain sensor made in the form of tenso-clasp with strain-element. The system provides the measurement of loads in 20 points in the range not more than 15 kg, and the accuracy of loads measurement ±3%. The mass of the loads measuring system is not more than 3.5 kg.

### FIELD OF THE INVENTION

The area of application of the invention is the development of special cloths for treatment in clinical conditions of the motor function disorders at child's cerebrally palsy, disturbances in vascular system of brain (strokes), and other diseases followed by disturbances of motor coordination both of arms and legs, and also for measuring and registration of loads on patient body created by the suit.

The task of the invention is to develop the medical suit of axial loading with the system of loads measuring and registration.

### PRIOR ART

Different constructions of the cloths for cosmonauts solving the tasks of human lightening and adaptation in weightlessness are known from the technique level (for example, RU2254272 C2, Jun. 20, 2005. U.S. Pat. No. 4,051,848 A, Oct. 4, 1977, RU2007146197/12, Dec. 14, 2007).

Their common disadvantage is the unsolved task of providing the load compensation and its control.

As a prototype the technical decision was chosen—prophylaxis load suit for cosmonauts consisting of knitted coverall with tensioning device (RU 2007146198 A 2007).

But the known suit does not provide the measurement, control and registration of the loads of tensioning buffers.

### SUMMARY OF THE INVENTION

The above disadvantages are removed by the use of the proposed invention the task and technical results of which is a creation of the medical cloths of axial loading for patient including the system of buffer loads control and measuring in clinic conditions.

The technical result can be achieved by use of the proposed medical suit containing the coverall made of one (at least) fabric layer, and tensioning device which is characterized by the providing of axial load on the musculoskeletal system of patient with use of buffers, and the suit is additionally provided with the system of loads measuring and registration containing the power source, tensodynamometers with intensifiers placed at the buffers in the fixed anatomic points, and analog-digital converter with USB cable, so the system can convert analog signals into digital ones and to transfer them to computer for operative analysis, imaging and registration. Tensodynamometers are connected with the buffers and made in the form of clasp with strain-element and intensifier through which the regulating tape of buffer is passed. The strain-element is fixed on the middle crosspiece of metallic clasp which is elastically deformed together with the strain-element during measuring of the buffer tension, and the DV voltage arises at the output which is proportional to the bending load value. To provide interface with ADC this voltage is intensified and enters the ADC as an analog signal. ADC converts this signal into digital format and transfers to computer. The R-program (Regulation program) for processing of the input signals from the ADC is installed to the computer from CD, and it provides the calibration of signals, their ranging and registration in the form of loads (in kg) effected on human wearing the suit.

The system provides the loads measuring and registration in 20 tensodynamometers in the range of 0 to 15 kg, and the accuracy of loads measurement ±3%.

### DETAILED DESCRIPTION OF THE INVENTION

The substance of the invention is the following:

The system of loads measurement is intended for the measuring and registration of loads on patient body created by the medical suit during its wearing in clinic conditions within the given time period of rehabilitation procedures.

The main mechanism of the suit effect on patient body is the creation of axial load on the musculoskeletal system with use of many buffers.

The suit provides an opportunity of loads variation in the large range: the total load can reach 40 or more kg and provides the organization of measurement, control and registration of the “human-suit” system state, performed in real time, and also the feedback for operative correction of the suit use regime during its wearing.

The use of the suit solves the following tasks:

1. Determination and creation of total axial load value optimal for a patient.

2. Operative change of load during rehabilitation of a patient with registration of the given loads in database.

3. Creation of the loads of specific muscle groups required for the treatment taking into account physiological characteristics of exact patient.

4. Realization of an opportunity to form the loading of patient muscles and skeleton symmetrically or asymmetrically, from the right—from the left, from the front—from behind.

The presence of feedback “physiological parameter—computer” allows creating of physical loads identical to current physiological status of patient, and tasks of exercises during treatment. For example, speed of exercises could be changed depending on the heartbeat frequencies, and sensitivity of the control channel could be changed depending on myogram level, and so on.

The proposed technical decision allows increasing the efficiency of the use of such medical cloths (suit of axial loading) thanks to computer support means.

Loading elements of the suit (elastic buffers) create axial load on the body and legs of patient. Strains from buffers tension can totally reach considerable value—up to 40 (or more) kg, but these strains are changed essentially depending on the position of body parts (respecting each other) which are “served” by this buffer. That is, strains from the buffers tension in the initial state (as a rule, this is vertical position with straightened body and legs) begin to change with appearance of angles in joints and have rapid dynamics while moving.

The objective registration of the strains in database supposes the presence of both static values and their dynamics with analysis of parameters accepted for dynamic processes and useful for assessment of patient state (for example, time of movement, correlation between bending and unbending, derivatives of movement: speed and acceleration, and so on).

The following example of the system of strains measurement can be given:

Each buffer of the suit has a strain sensor made in the form of tenso-clasp through which the load strap passes (at the coverall this is regulating tape of the buffer). The shape of strain sensor in the form of plain clasp (20×20×2 mm) well combines with the tensioning element of the suit not disturbing its constructive design.

The strain element itself is fixed on the middle crosspiece of the clasp which is elastically deformed together with strain-element during changing of the buffer tension. The deformation of the strain-element causes the change of signal in it which is proportional to the strain in the buffer. The strain value in the buffers is determined according to the rating curves of the respective strain-sensors.

It is supposed to use the strain-element like KF (Constant foil which the element is made of) 5 P (rectangular tensoresistor having base 5 mm) which is powered from the power source with voltage 5 V. The signal is given to the ADC through the intensifier, and then to the computer for processing under the special program with visual operative imaging and printing.

A lot of data processing programs can be used.

One of the examples is a system of the medical suit control automatization assuming:

1. Objectification of information on technical state of the suit;

2. Operative getting in real time of the integral parameters of the suit loading;

3. Intensification of the suit loading data processing.

The parameters determining the way of the suit using are to be registered: maximal strains in the buffers, integral estimate of the strain of several buffers, capacity of the buffers as a qualitative characteristic of the energy consumption during exercises, critical characteristics determining the excess of the permissible range of the technical signal values.

The load measuring system with use of 20 tensodynamometers would provide the measuring of the loads created by the medical suit on the human body, converting of the analog signals into digital format, and their transfer to the computer for operative analysis, imaging in real time and registration.

The load measuring system should have the following technical characteristics:

a) to provide load measuring in 20 points in the range 0÷15 kg;

b) accuracy of loads measurement ±3%;

c) mass of the loads measuring system—not more than 3.5 kg;

The specified life should be not less than 1000 hours.

Specified working time should be not less than 5 years.

The guaranteed number of connections-disconnections should be not less than 250.

Electric resistance of wires covering of the initial supply circuits relative to body should be not less than:

a) 20 Mohm under RH 20-90% and temperature 10-25° C.

σ) 1 Mohm under RH 95±3% and temperature 20° C.

Electric isolation of the wires of initial supply circuits relative to body should stand the test voltage 500 B of the continuous current within 1 minute.

Control of work capacity of the ADC in the load measuring system should be provided by the integral system of control and diagnostics.

Software for the system of the medical suit load measuring:

Work capacity of the load measuring system is controlled by use the computer with installed operational system Windows XP/VISTA.

Computer software works in Windows XP/VISTA and allows performing the following functions:
Getting data from tensodynamometers with the frequency 20 Hz.
Taking into account the initial shift of output zero from each sensor.
Displaying at the computer monitor the tables with the values of current loads from all buffers separately and totally according to the anatomic body parts (left shoulder, right shoulder, chest, back, and so on).

Form view of the software should be reflected at the display in the form of scheme of sensors disposition at the medical suit, table with current values of loads for all buffers and groups of buffers (28 meanings in total), and also short comment to the treatment session: names of patient and operator, date, time of load registration, name of sensor position.

1. Medical suit for axial loading with automatic control system containing coverall made of at least one fabric layer, and tensioning device, characterized in that the tensioning device provides creation of axial loading on the musculoskeletal system with use of buffers, and in that said coverall is additionally provided with a system for load measurement and registration containing power source, tensodynamometers with intensifiers placed in the fixed points, and analog-digital converter with USB cable, so that the system can convert analog signals into digital ones and to transfer them to a computer for operative analysis, imaging and registration.

2. Suit according to claim 1, characterized in that every tensodynamometer is made in the form of metallic clasp with strain-element and intensifier through which the regulating tape of buffer is passed.

3. Suit according to claim 2, characterized in that strain-element is fixed on the middle crosspiece of metallic clasp which is elastically deformed together with strain-element during measuring of the buffer tension.

4. Suit according to claim 1, characterized in that the system provides measurement of loads in 20 points in the range of 0 to 15 kg.

5. Suit according to claim 1, characterized in that the system provides the accuracy of loads measurement ±3%.

6. Suit according to claim 1, characterized in that the mass of the loads measuring system is not more than 3.5 kg.
